(19) United States
(12) Patent Application Publication
(10) Pub. No.: US 2017/0086675 A1
(43) Pub. Date: Mar. 30, 2017

(54) QUANTITATIVE TISSUE PROPERTY MAPPING FOR REAL TIME TUMOR DETECTION AND INTERVENTIONAL GUIDANCE

(71) Applicant: THE JOHNS HOPKINS UNIVERSITY, Baltimore, MD (US)

(72) Inventors: Xingde Li, Ellicott City, MD (US); Alfredo Quinones-Hinojosa, Bel Air, MD (US); Elliot McVeigh, Timonium, MD (US); Kaisorn Chaichana, Baltimore, MD (US); Carmen Kut, Baltimore, MD (US); Jiefeng Xi, Baltimore, MD (US)

(21) Appl. No.: 15/128,152

(22) PCT Filed: Mar. 25, 2015
(86) PCT No.: PCT/US2015/022432

§ 371 Date: Sep. 22, 2016

Related U.S. Application Data
(60) Provisional application No. 61/970,104, filed on Mar. 25, 2014.

(51) Int. Cl. A61B 5/00; A61B 90/20

(52) U.S. Cl. CPC A61B 5/0073; A61B 5/00; A61B 5/0066; A61B 5/0086; A61B 5/7425; A61B 90/20

(57) ABSTRACT

The present invention is directed to a method for real-time characterization of spatially-resolved tissue optical properties using OCT/LCI. Imaging data are acquired, processed, displayed and stored in real-time. The resultant tissue optical properties are then used to determine the diagnostic threshold and to determine the OCT/LCI detection sensitivity and specificity. Color-coded optical property maps are constructed to provide direct visual cues for surgeons to differentiate tumor versus non-tumor tissue. These optical property maps can be overlaid with the structural imaging data and/or Doppler results for efficient data display. Finally, the imaging system can also be integrated with existing systems such as tracking and surgical microscopes. An aiming beam is generally provided for interventional guidance. For intraoperative use, a cap/spacer may also be provided to maintain the working distance of the probe, and also to provide biopsy capabilities. The method is usable for research and clinical diagnosis and/or interventional guidance.

### CROSS REFERENCE TO RELATED APPLICATIONS

This application claims the benefit of U.S. Provisional Patent Application No. 61/970,104 filed Mar. 25, 2014, which is incorporated by reference herein, in its entirety.

### GOVERNMENT RIGHTS

This invention was made with government support under R01EB007636, R01CA120480, and R01NS070024 awarded by the National Institutes of Health. The government has certain rights in the invention.

### FIELD OF THE INVENTION

The present invention relates generally to medical imaging. More particularly, the present invention relates to a method for Optical Coherence Tomography (OCT) or low coherence interferometry (LCI) imaging based tumor detection and interventional guidance.

### BACKGROUND OF THE INVENTION

Approximately 1,665,540 new cancer cases and 585,720 cancer deaths occur annually in the United States. Surgery is the primary method of treatment for most isolated solid cancers and often plays a role the prolongation of survival. Previous studies have shown that there is a critical need to cut out more tumor during cancer surgery, especially at the infiltrative tumor boundaries. This clinical need can be applied to multiple cancer types such as head and neck cancer, brain cancer, breast cancer, oral cancer, soft tissue sarcomas and gastrointestinal cancer to name a few. For the following, we will use brain cancer as an example but it is understood that the present invention is not limited to brain cancer.

Imaging technologies have played an increasingly significant role in helping achieve optimal tumor tissue removal. However, there are several shortcomings with existing imaging technologies in the operating room. For example, surgical navigations based on pre-operative MRI is the current standard of care for brain cancer, but causes large positional errors from the patient's motions e.g. breathing and heartbeat. Intra-operative MRI provides better resolution and accuracy, but does not provide real-time continuous guidance; it is also time consuming and often costs millions of dollars per unit, which only few hospitals can afford. Ultrasound is portable and low-cost, but its use in the operating room is limited for certain cancer applications due to insufficient tissue contrast and resolution. Finally, fluorescence imaging often involves the use of an oral or intravenous contrast agent, and the heterogeneous uptake.

Optical Coherence Tomography (OCT) or low coherence interferometry (LCI) imaging have significant advantages over the aforementioned medical imaging technologies in detecting tumor during the surgery. OCT and/or LCI are non-invasive, high-resolution optical imaging technologies capable of real-time imaging of tissue microanatomy with a few millimeter imaging depth. OCT and/or LCI function as a form of “optical biopsy”, capable of assessing tissue microanatomy and function with a resolution approaching that of standard histology but without the need for tissue removal. In addition, optical properties derived from OCT or LCI images can be used to quantitatively analyze tissues and provide real-time and direct visual guidance for tumor resection. As a result, there is a need in the art for a method of OCT/LCI imaging for tumor detection and interventional guidance.

### SUMMARY OF THE INVENTION

The foregoing needs are met, to a great extent, by the present invention which provides a method for real-time characterization of spatially resolved tissue optical properties for one-dimensional (1D), two-dimensional (2D), or three-dimensional (3D) imaging over a given tissue derived from OCT or LCI imaging data. The method also includes generating a quantitative, color-coded, and high-resolution optical property map. Additionally, the method includes establishing a diagnostic threshold for optical properties used for differentiating tumor from non-tumor with high sensitivity and specificity.

In accordance with an aspect of the present invention, the method includes programming the steps of the method on non-transitory computer readable medium/media. This method includes a programming method to acquire, process, display and stores optical properties of tissues in real-time and in high-resolution. This method includes mechanisms to analyze the depth-dependent imaging data using exponential and Frequency-domain fitting methods for ultrafast and reliable characterization of optical properties with high computational efficiency and accuracy. This method includes mitigating the influence of the depth-dependent effects of the beam profile by creating phantoms with known optical properties and by calibrating the OCT or LCI imaging data with the phantom imaging data. This method includes algorithms optimized for tissue characterization including speckle, motion and blood artifact identification and minimization, and tissue surface identification from the blood pool. This method includes the systematic and quantitative analysis of cancer tissues in real-time using the imaging data obtained. The method includes using optical property values (such as optical attenuation, backscattering, scattering and absorption to name a few, and the combination of any of these parameters) to determine areas of tumor versus areas of non-tumor. This method includes providing direct visual cues using the color-coded map for the surgeon to differentiate tumor from non-tumor tissue for the imaged tissues (for 1D, 2D and 3D scanning) and combining the OCT or LCI image with the overlaid optical property map and/or Doppler information to identify critical structures such as blood vessels, avoiding potential injury during surgical interventions. This method includes varying the imaging beam spot size to control transverse resolution and the imaging/display speed.

In accordance with another aspect of the present invention, the present invention is also directed to a system and method integrated with the optical imaging device for tracking the position and orientation of the imaging device, imaging beam and the imaging area on the target in real-time (as identified in a resultant map) and with an aiming beam for visualization of the region of interest on the target and for interventional guidance. The method includes the use of caps/spacers to maintain the working distance of the compact imaging probe and to provide additional tissue resection capabilities to remove the exact region of interest which was imaged. This facilitates the removal of cancerous tissues during interventional guidance; in addition, the removed tissue can be submitted for histological processing, thereby providing accurate imaging-histological correlations for basic science/clinical research purposes. This method includes the implementation of graphics processing unit (GPU)-based or field-programmable gate array (FPGA)-based parallel processing algorithms for optimal computational efficiency and real-time acquisition, processing and displays of tissue optical properties, structures and blood flow.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates the overall schematics of the present invention including the Optical Coherence Tomography (OCT) or Low Coherence Interferometry (LCI) imaging hardware and software. First, the OCT/LCI light source is directed to hardware components such as the compact imaging probe and an interferometer. The resultant OCT/LCI and calibration signal is then transferred through a digitizer to the computer interface for data acquisition, processing, display and storage. Notably, the position and orientation of the OCT/LCI imaging probe can be tracked using existing devices (e.g. EM tracker, Polaris tracker and surgical microscopes to name a few). In addition, the OCT/LCI imaging display can be integrated with displays from other intraoperative image guidance systems (e.g. surgical microscope and MRI/CT surgical navigational systems, to name a few). Finally, the present invention also includes the use of an aiming beam (to visualize the targeted imaging area) and the use of disposable imaging caps (which can be used as a spacer to maintain the working distance, but can also be activated as a biopsy cap to resect the exact imaged tissue volume).

FIGS. 2A-2C illustrate an example of an OCT/LCI imaging system. In this particular example, we presented a home-built swept source optical coherence tomography system (SS-OCT) imaging system, a 2D scanning compact imaging probe, and a schematic of the SS-OCT imaging system. BD: balanced detector; CIR: circulator; CL: collimating lens; DAQ: data acquisition; MZI: Mach-Zehnder Interferometer; OC: Optical Coupler

FIG. 3 illustrates exemplary images of an OCT/LCI imaging system. In this particular example, we presented results obtained from cross-sectional OCT images for freshly resected human brain cancer tissues. The results showed tumor specific characteristics e.g. necrosis (N) and hypercellularity (H) in high-grade brain cancer. Similarly, the results revealed microcyst formation (black arrows) in low-grade brain cancer. In contract, non-cancer white matter tissues—obtained from resected tissues from a seizure patient (control) and from the resection margin of a brain cancer patient—appeared homogeneous with high attenuation on OCT images. Scale bars: 500 μm.

FIG. 4 illustrates a schematic diagram and associated equations for the algorithms used to evaluate the relevant tissue optical properties, according to an embodiment of the present invention. In this particular example, we presented the equations used to evaluate the tissue optical attenuation. The OCT/LCI intensity data is depth-dependent and can be described by an exponential equation where I is the intensity data, z is the depth, k is a system constant, μ_{bs} is backscattering coefficient, h(z) is the geometric factor of the imaging beam, and μₜ is the attenuation coefficient. To minimize the depth-dependent influence of the beam profile, phantoms were created with known optical properties and the tissue imaging data were calibrated with the phantom imaging data. Then, the optical attenuation values were obtained using one of two methods: 1) a traditional exponential intensity fitting method (or linear fitting of the logarithm of the intensity), where C is a constant, μ_{t,b} and μ_{t,p} are the attenuation coefficient of the biological tissue and that of the phantom, respectively; 2) a frequency-domain (FD) algorithm which computes the ratio between two harmonic components from the Fourier transform of the imaging data to obtain the required components. Here, κ is the spatial frequency, while |F(κ=0)| and

$$F ( κ = \frac{2 π}{N \cdot Δ z} )$$

are the zeroth and first harmonic components, respectively.

FIG. 5A illustrates a flow diagram of the methods used to detect the beginning of the tissue depth regardless of uneven surfaces, respiratory/pulsatile motion, and the presence of accumulating blood pools. FIG. 5B illustrates an exemplary image and graphical view of when it is necessary to separate any accumulating blood pools from the actual tissue surface. I(z): depth-dependent OCT/LCI intensity signal and Iₘₑₐₙ(z): laterally averaged OCT/LCI Intensity signal.

FIGS. 6A-C illustrate flow diagrams of the methods used in a double-blinded study to establish the training and validation datasets. The training dataset is used to establish an optical diagnostic threshold to detect tumor versus non-tumor tissues based on the desired sensitivity/specificity criteria. The validation dataset is used to compute the OCT/LCI detection sensitivity and specificity using the chosen optical diagnostic thresholds.

FIGS. 7A-B illustrates image examples on how an imaging user can toggle different modes of imaging data (e.g. structural imaging data, optical property map and Doppler information, or any combination of these data) on and off for the desired image display configurations. FIG. 7A illustrates an example when the 3D structural imaging data is overlaid with an en face optical attenuation map; FIG. 7B illustrates an example when the 3D imaging data is overlaid with the Doppler blood flow map.

FIG. 8A illustrates a schematic diagram of one example on how the position and orientation of the OCT/LCI compact imaging probe can be tracked using an existing system (e.g. infrared tracker, electromagnetic tracker or surgical microscopes). FIG. 8B illustrates on example on how the OCT/LCI infrared laser source can be coupled with a visible aiming beam to visualize the imaged area on the tissue surface.

FIG. 9 illustrates a schematic diagram of how disposable imaging caps can be used intraoperatively. Before imaging, the cap works as a spacer to maintain the working distance between the compact OCT/LCI probe and the region of interest (ROI) which was being imaged as part of the intact tissue surface. Immediately after imaging, the imaging cap acts as a biopsy device to resect the imaged ROI from the tissue surface. Following biopsy, the imaging cap (containing the resected tissue) will be detached from the OCT/LCI probe and sent to histology. A new imaging cap will then be activated and/or attached to the image probe.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

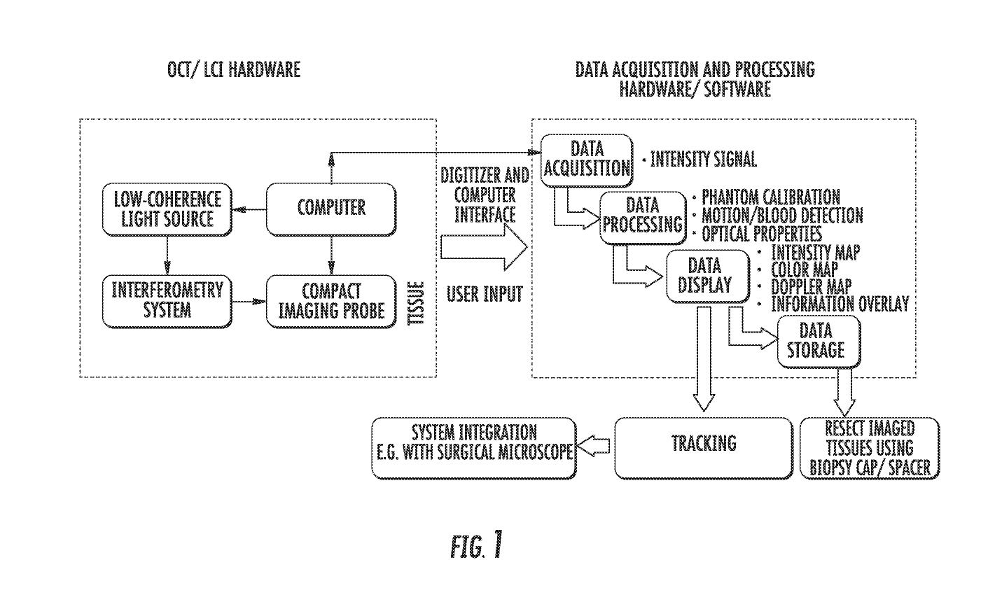

The present invention is directed to a method for and a non-transitory computer readable medium programmed to enable real-time characterization of spatially resolved tissue optical properties with excellent spatial resolution over a given tissue volume. The overall schematics of the present invention has been summarized in FIG. 1. Please note that LCI and OCT will be used interchangeably, herein.

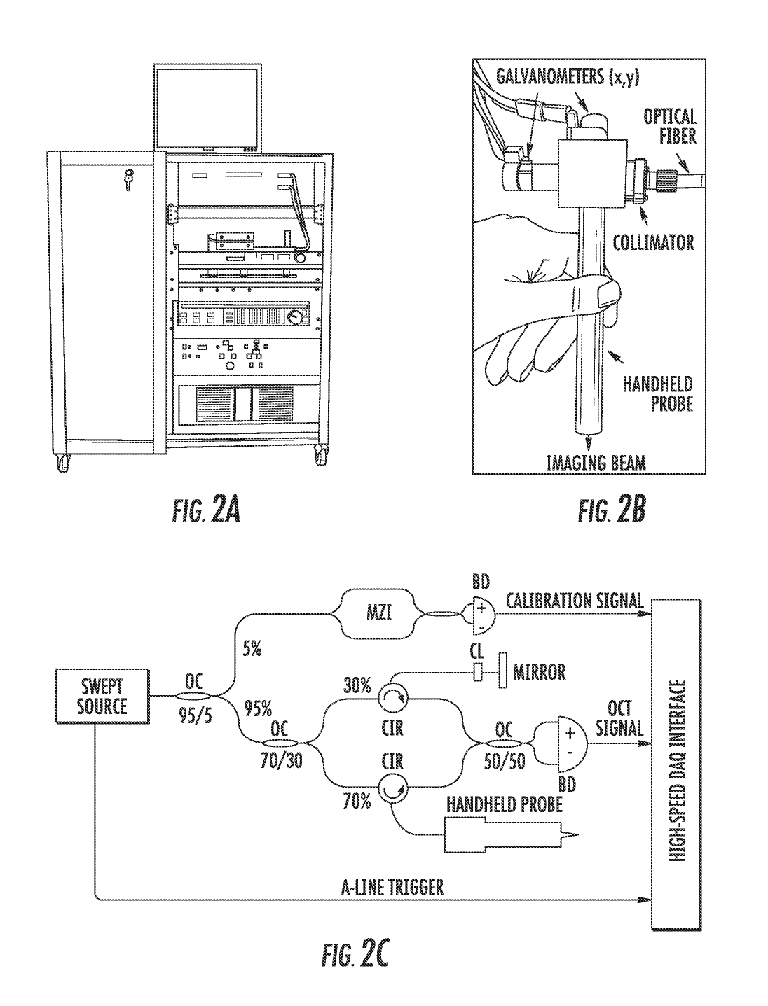

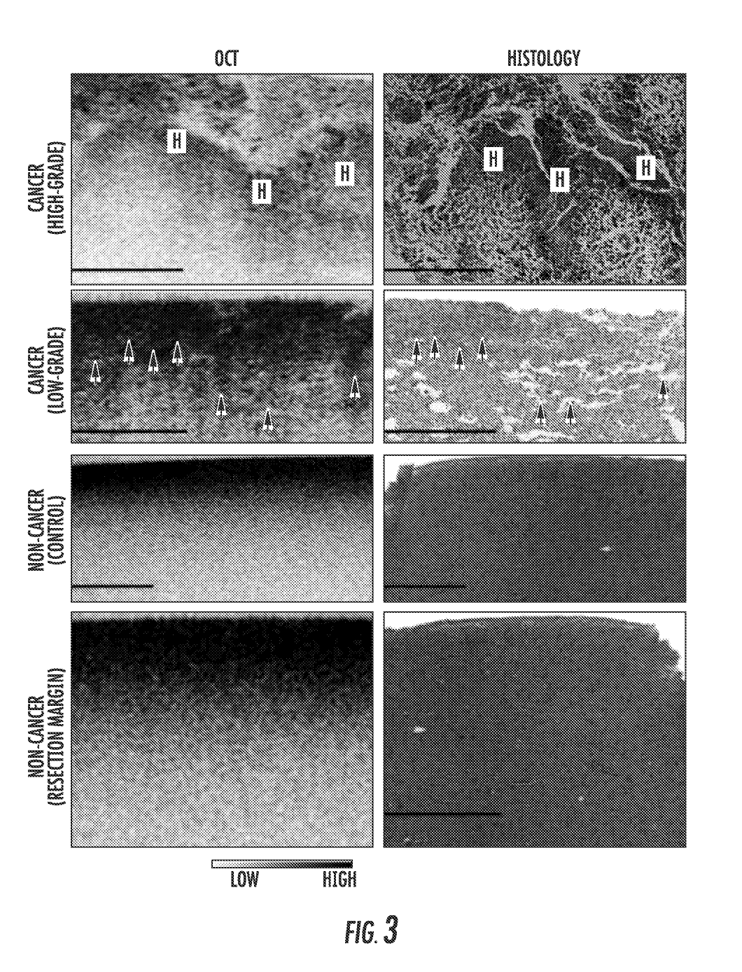

Preliminary human ex vivo studies: one application of the concepts disclosed herein is to use OCT or LCI imaging and any derived optical properties to detect cancerous versus non-cancerous tissues. To determine whether OCT and LCI can be used to detect cancerous tissues, extensive study on ex vivo tissues were performed for freshly resected human tissues resected from cancer patients in the operating room. In this study, we collected human tissues from brain cancer patients for demonstration purposes (although the same methods can be applied for many other cancer types such as breast cancer, oral cancer, gastrointestinal cancer and skin cancer to name a few). These human tissue specimens were imaged using a homebuilt optical imaging system (generally consistent with the OCT and/or LCI system illustrated in FIGS. 2A-2C). Representative optical images with the corresponding histological images obtained using microscopic techniques were illustrated in FIG. 3. Features that can be identified in the OCT image of FIG. 3 and the corresponding histological image in FIG. 3 include normal non-cancer white matter tissues and cancerous tissues (containing features such as necrosis, areas of hypercellularity and the presence of microcysts). Significantly, such features can be identified in the optical images and correlated well with histology.

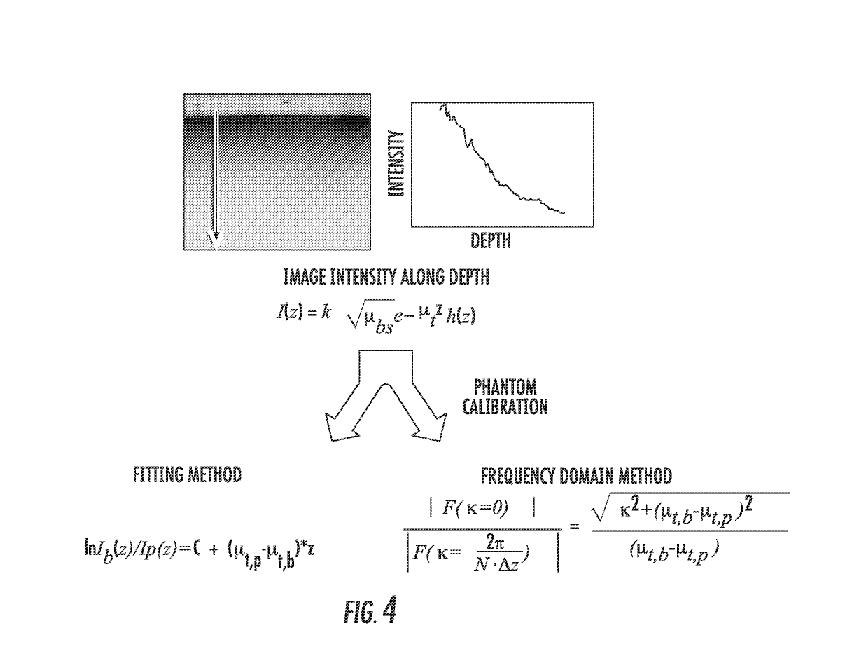

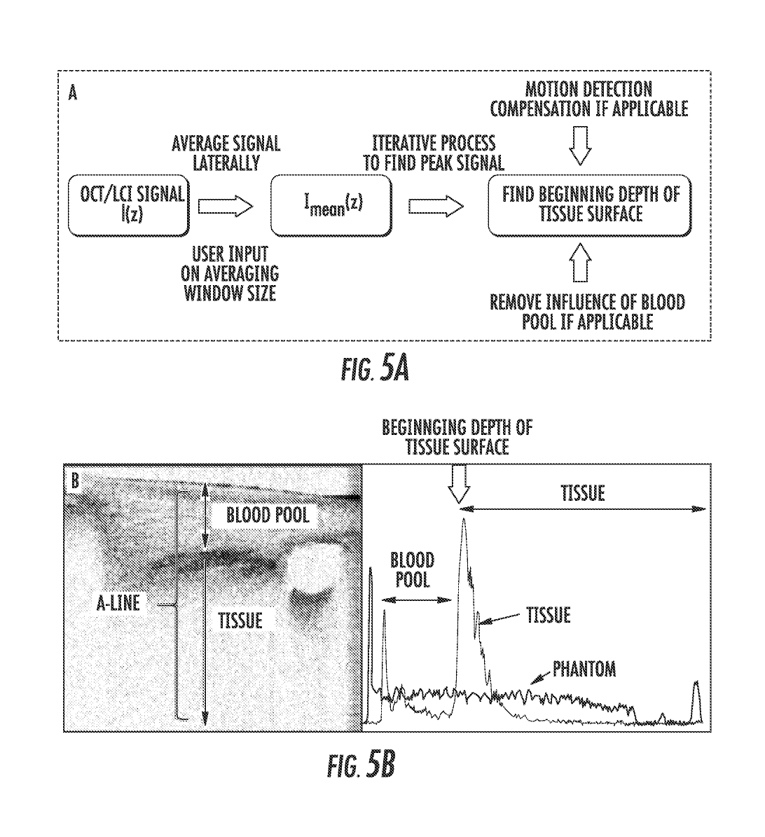

Additionally, optical properties were computed for both tumor and non-tumor specimens. To accomplish this, specific algorithms were developed to analyze, average and fit the optical imaging data. FIG. 4 illustrates the schematics and associated equations for the algorithms used, namely a traditional exponentially fitting method and a novel frequency-domain (FD) algorithm which computes the ratio between two harmonic components to obtain the required components. Additionally, phantoms were created with known optical properties (using media such as gelatin and resin, and using scatters/absorbers such as silicon oxide or titanium oxide/Indian ink, to name a few); using Mie theory, we can accurately predict the optical properties for these phantoms. These optical properties include attenuation, backscattering and scattering and absorption coefficients, to name a few. Importantly, optical properties are difficult to evaluate using traditional methods because of the influence of depth-dependent effects of the beam profile; in our study, we calibrated the tissue imaging data with the phantom imaging data in order to mitigate such influence. To optimize our algorithms for ex vivo versus in vivo imaging of human tissues, FIG. 5A illustrates the methods used to detect the beginning of the tissue depth regardless of uneven surfaces, respiratory/pulsatile motion, and the presence of accumulating blood pools. FIG. 5B illustrates an example when it is necessary to separate any accumulating blood pools from the actual tissue surface. To summarize, FIGS. 4-5B illustrate the programming method to acquire and process optical imaging data and to obtain relevant optical property values for a tissue specimen.

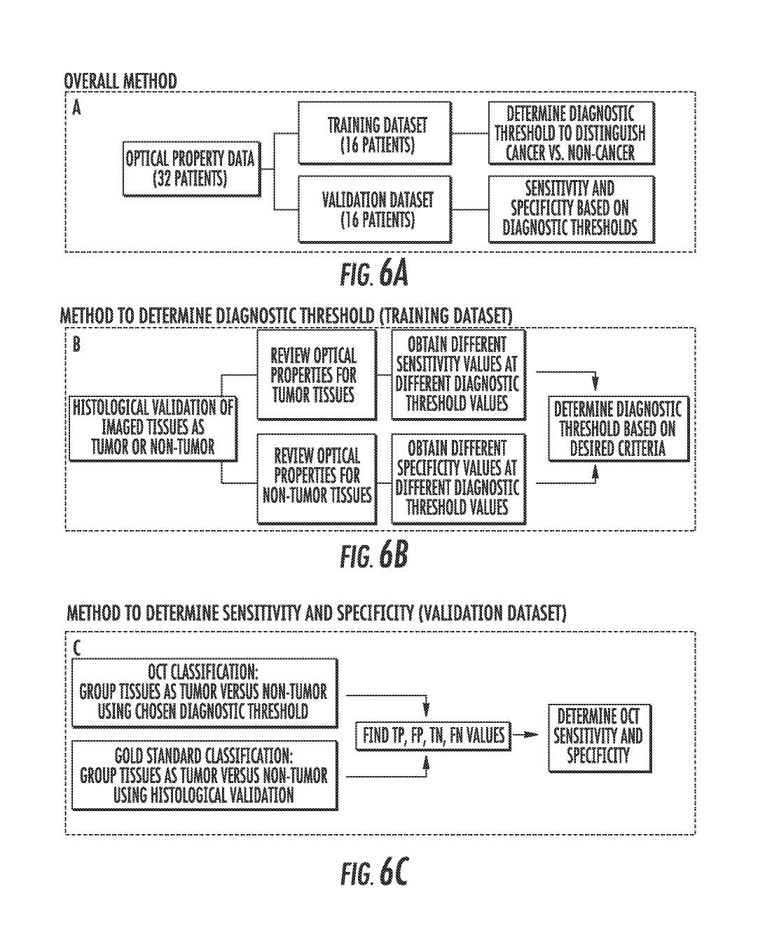

Once the optical imaging system has captured the imaging data and the associated optical property values of the tissue specimens are analyzed, these specimens are submitted to histological processing and validation. FIG. 6A illustrates how tissues from 32 patients were divided into 2 independent datasets: 1) a training set with 16 patients and 2) a double-blinded validation dataset with 16 patients.

In the training dataset, the histological slides of each tissue specimen were reviewed by a pathologist, who classifies a tissue specimen as either cancer or non-cancer. Based on these results, a diagnostic optical threshold was established to distinguish tumor versus non-tumor; for example, tissues with optical properties above the threshold value are classified as non-cancer, and tissues with optical properties below the threshold value are classified as cancer. FIG. 6B illustrates how diagnostic optical thresholds are determined tissues by comparing the optical properties of a tissue specimen with its corresponding histological diagnosis (cancer or non-cancer). Notably, the diagnostic optical threshold can be configured and adjusted according to the desired sensitivity and specificity criteria.

In the validation dataset, both the imaging user and the pathologist were blinded to the patient's clinical diagnosis (e.g. control patients with normal histology, or cancer patients). FIG. 6C summarizes the method used to determine the sensitivity and specificity from the validation dataset. First, the diagnostic optical threshold (obtained from the training set) was used to determine the optically-based diagnosis (on whether a tissue specimen is classified as cancer or non-cancer) using OCT or LCI imaging. Second, the pathologists reviewed the histological slides obtained from the tissue specimens and determine the histologically-based diagnosis (on whether a tissue specimen is classified by histology as cancer or non-cancer). Finally, the optical detection sensitivity and specificity of this study was computed by comparing the optically-based diagnoses with the histological-based diagnoses.

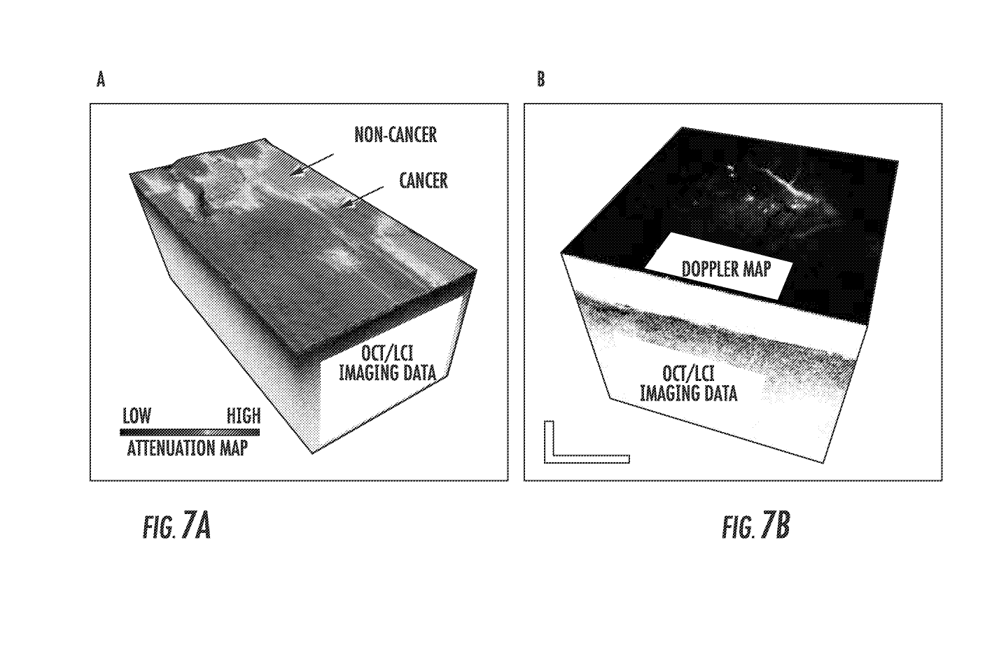

After determining the optimal diagnostic threshold, a color-coded optical property map is constructed and displayed over the 1D, 2D or 3D optical imaging data to differentiate cancer from non-cancer for the given tissue specimens. The color-coded map can provide direct visual cues for the surgeon to differentiate tumor from non-tumor tissue for the imaged tissue. In addition, the user can toggle different modes of imaging data (e.g. structural imaging data, optical property map and Doppler information, or any combination of these data) on and off for the desired image display configurations. FIGS. 7A and 7B illustrate some examples for these image display configurations. Importantly, the above imaging modes can also be combined and overlaid over one another to provide efficient information display and also to identify critical structures such as blood vessels, thus avoiding potential injury during surgical interventions. Importantly, these image displays can also be further configured based on the user's preference on window size, optical property resolution, imaging speed and other parameters. The method can be used for research and clinical diagnosis and/or interventional guidance. Pathologically-confirmed brain cancer tissues have significantly lower optical attenuation values at both the cancer core and infiltrated zones, when compared with non-cancer. Using these optical threshold values, our method achieved ≧90% sensitivity and ≧80% specificity at the specified optical property (e.g. attenuation, backscattering, scattering, absorption, and any combination of these parameters). Furthermore, this threshold is usable to confirm the intraoperative feasibility of performing OCT or LCI-guided surgery using a mammalian model harboring human cancer (with both commercial and patient-derived cell lines). Quantitative, spatially resolved, and color-coded optical property map derived from OCT or LCI measurements can therefore be used for differentiating tumor from non-tumor tissues. Its intraoperative use may facilitate safe, extensive resection of infiltrative cancers and may lead to safer surgeries with improved outcomes.

In addition, the present invention also includes the development of graphics processing unit (GPU)-based and/or field-programmable gate array (FPGA)-based parallel processing algorithms which enabled efficient and real-time image acquisition, processing, display and storage of the optical imaging data as well as any associated optical properties. These software algorithms can be further configured based on any desired parameters including but not limited to imaging speed, desired display and computation format, and storage specification.

An embodiment according to the present invention also includes a non-transitory computer readable medium programmed to receive 1D, 2D or 3D OCT and/or LCI imaging data. Along with the optical imaging data, a quantitative, color-coded, and high-resolution optical property map is generated. The non-transitory computer readable medium is programmed to establish a threshold for optical properties and used for differentiating tumor from non-tumor with high sensitivity and specificity.

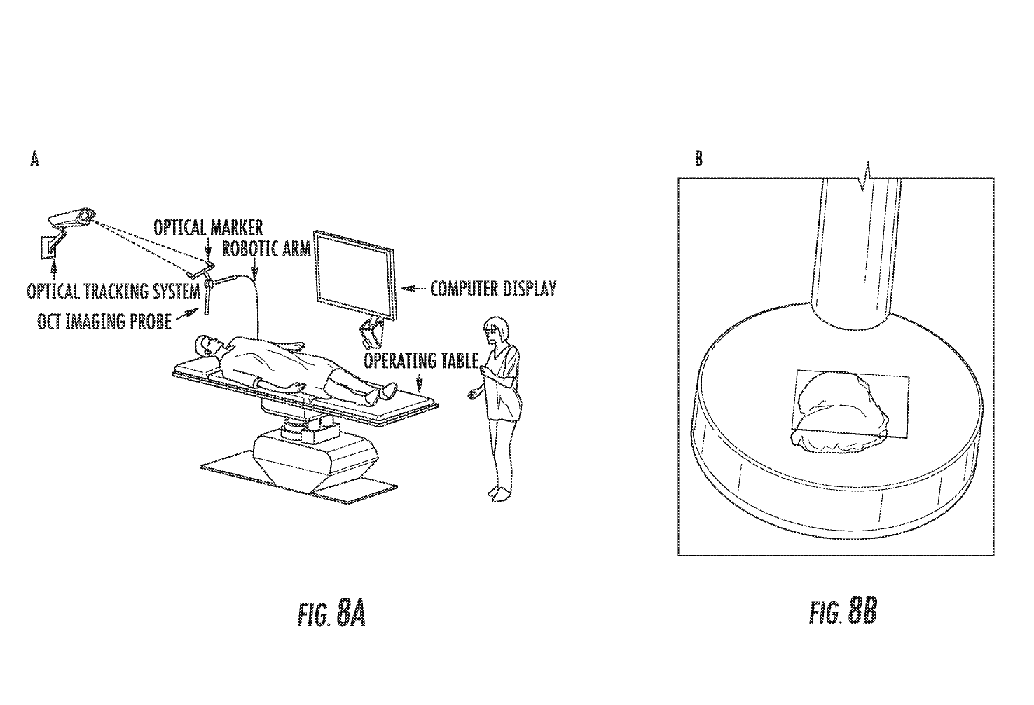

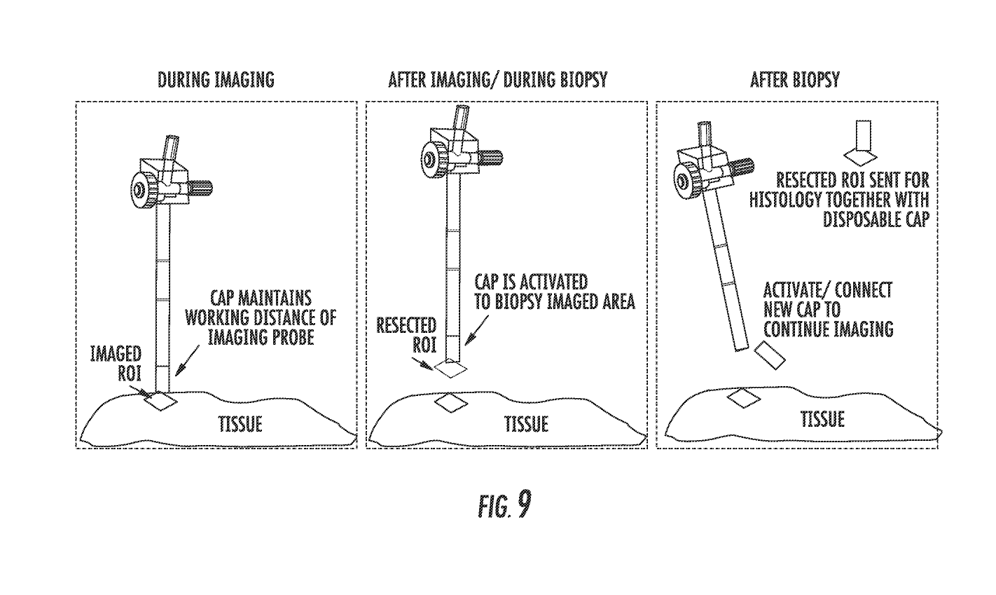

In addition, the invention can include a single non-transitory computer readable medium or two or more non-transitory computer readable media working together in parallel to process the 1D, 2D or 3D optical imaging data. This setup allows for quick extraction of optical properties over a given tissue's region of interest. The non-transitory computer readable medium can reside on the OCT and/or LCI imaging system or a separate computing device, server, or other computer networked either over hard wire or wirelessly to the optical imaging system for tracking regions of interest in real-time (as identified by the color-coded optical property map) with an aiming beam for interventional guidance. These tracking methods include but are not limited to the use of existing commercial tracking systems (e.g. infrared tracking or electromagnetic tracking of specific markers), or the integration of the optical imaging system to the surgical microscope (both conventional and stereoscopic). These tracking systems will be integrated with an OCT or LCI imaging system for tracking regions of interest in real-time and by overlaying multiple video/image feeds for optimal display of information. Examples of aiming beams include but are not limited to the use of laser sources, LED lights and other methods to visualize the OCT scanning region/field of view. FIG. 8A illustrates one example schematic for tracking the position and orientation of the imaging device, imaging beam and imaging area on the target in real-time (as identified in a resultant map). In addition, FIG. 8B also illustrates one example of the use of aiming beams used to visualize the region of interest on the target and also for interventional guidance. In addition to tracking and aiming beams, our invention can also include a cap/spacer to maintain the working distance of the imaged tissue surface from the compact imaging probe, and also to provide additional tissue resection capabilities to remove the exact region of interest which was imaged. As illustrated in FIG. 9, this method can be used to remove cancerous tissues during interventional guidance, and also for accurate imaging-histological correlations for basic science/clinical research purposes.

Finally, while the present invention is discussed with respect to the example of detection and interventional support for brain tumors, the same methodology can be used for tumor detection or interventional guidance in other organs or systems for both research and clinical use (including breast cancer, oral cancer, head and neck cancer and skin cancer to name a few).

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A method for real-time characterization of spatially resolved tissue optical properties over a given tissue volume to differentiate tumor from non-tumor, comprising the steps of:
acquiring, processing, displaying and storing imaging data;
analyzing the imaging data for optimal tissue characterization including speckle, motion and blood artifact identification and minimization, and tissue surface identification from blood pool;
analyzing the data using exponential and Frequency-domain fitting methods for characterization of optical properties;
establishing a diagnostic threshold for optical properties used for differentiating tumor from non-tumor tissue based on selected detection sensitivity and specificity criteria;
generating a quantitative, color-coded, and high-resolution optical property map for the given tissue volume, which will provide direct visual cues to differentiate tumor from non-tumor tissues with the imaging data; and
superimposing the quantitative, color-coded, and high-resolution optical property map onto the imaging data to enable data display.

2. The method of claim 1, further comprising using one selected from a group consisting of one dimensional (1D), two dimensional (2D), and three dimensional (3D) imaging data.

3. The method of claim 1, further comprising using one selected from a group consisting of optical coherence tomography and low coherence interferometry.

4. The method of claim 1, further comprising programming the steps of the method on one or more non-transitory computer readable medium (media).

5. The method of claim 1, further comprising averaging and reorganizing imaging data for optimal computational efficiency and real-time acquisition, processing and displaying of the imaging data and resultant color-coded maps.

6. The method of claim 1, further comprising configuring beam spot size of acquiring imaging data to control the transverse resolution and the imaging/displaying speed.

7. The method of claim 1, further comprising using one selected from a group consisting of high-speed photo detector, digitization card, GPU and FPAG, parallel algorithms and high-speed digital storage device(s) to provide optimal computational efficiency and real-time acquisition, processing and display of OCT imaging data and the tissue optical properties, structure and blood flow.

8. The method of claim 1, further comprising mitigating influence of depth dependent effects of the beam profiles by calibrating the imaging data with phantom data.

9. The method of claim 1, further comprising processing imaging data for speckle reduction and then analyzing the imaging data for optical property quantification by one selected from a group consisting of fitting intensity decay (or the logarithm of the intensity) versus depth over a given depth range of interest and using a Frequency domain harmonics analysis method, wherein a ratio between two harmonic components of a Fourier transformed intensity signal is identified.

10. The method of claim 1, further comprising coding the optical property map with color, and overlaying the optical property map with Doppler information to identify critical structures such as blood vessels, avoiding potential injury during surgical interventions.

11. The method of claim 1, further comprising equipping an optical imaging device with a system and method for tracking the position and orientation of the imaging device, imaging beam, and imaging area on the target in real-time, as identified in a resultant map.

12. The method of claim 1, further comprising integrating an aiming beam for visualization of the region of interest on the target and for interventional guidance.

13. The method of claim 1 further comprising differentiating tumor tissue from non-tumor with quantitative analysis and color coding.

14. The method of claim 1 further comprising using optical parameters such as attenuation, backscattering, scattering and absorption or the combination of any of these parameters to distinguish cancerous tissue from non-cancerous tissue.

15. The method of claim 1 further comprising using the optical property map for interventional guidance.

16. The method of claim 1, further comprising configuring an imaging system for acquiring the imaging data and a compact imaging probe to provide the desired resolution, imaging speed, probe length and other parameters for optimal use in a given application.

17. The method of claim 14, further comprising maintaining a working distance of a compact imaging probe using a cap/spacer, and to providing additional tissue resection capabilities to remove the exact region of interest which was imaged, such that removal of cancerous tissues during interventional guidance is facilitated and removed tissue can be submitted for histological processing, thereby providing accurate imaging-histological correlations.

18. The method of claim 2, further comprising using the two or more non-transitory computer readable mediums working in parallel.

19. The method of claim 5, further comprising acquiring, processing and displaying imaging data points and frames in high-speed.

20. The method of claim 6, further comprising creating phantoms with known optical properties.

21. The method of claim 9, further comprising configuring the system for tracking to control an OCT field of view and scanning mechanisms.

22. The method of claim 9, further comprising configuring the system for tracking to integrate an OCT or LCI imaging beam with other imaging devices to provide multi-modal information about the target with or without co-registration.
